(19) United States
(12) Patent Application Publication
(10) Pub. No.: US 2007/0225364 A1
(43) Pub. Date: Sep. 27, 2007

(54) Use of bismuth subgallate and borneol in treating melanoma

(75) Inventors: James J.T. Chen, Taipei (TW); Black J.B. Chen, Taipei (TW)

Correspondence Address: LADAS & PARRY, 26 WEST 61ST STREET, NEW YORK, NY, 10023, US

(73) Assignee: HEDONIST BIOCHEMICAL TECHNOLOGIES CO., LTD.

(21) Appl. No.: 11/385,876

(22) Filed: Mar. 21, 2006

(51) Int. Cl. A61K 31/29; A61K 31/045

(52) U.S. Cl. 514/503; 514/729

(57) ABSTRACT

A new use of bismuth subgallate and borneol for treating melanoma by the reduction of the number of melanoma cells and/or the inhibition of the proliferation of melanoma cells is provided. A use of bismuth subgallate and borneol for inhibiting the migration of melanoma cells is also provided.

### TECHNICAL FIELD

This invention relates to a novel use of bismuth subgallate and borneol in the reduction of the number of melanoma cells and the inhibition of the proliferation and the migration of melanoma cells.

### BACKGROUND OF THE INVENTION

Melanoma is a kind of malignant skin cancer, which is a relatively common neoplasm. There are three main types of malignant skin cancers, including malignant melanoma, basal cell carcinoma and squamous cell carcinoma. Basel and squamous cell carcinoma are often grouped together and referred to as non-melanoma skin cancer. The main factor to induce skin cancer is to be exposed to ultraviolet light of sunshine. Thus, skin cancer is very common in areas with strong sunlight, like Australia, and is less common in people with dark skin. The incidence of malignant melanoma has doubled every ten years since the 1950's and it is predicted that by the year 2010, the ratio of the number of patients to the population in the UK will approach 1 in 50. Unlike other cancers, metastatic melanoma is virtually untreatable by the normal methods of cancer treatment such as chemotherapy, radiotherapy or interferon. Furthermore, the migration of melanoma cells is often observed. Sixty-six percent of patients with metastatic melanoma die within five years. Thus, alternative, effective methods for treating malignant melanoma are urgently needed.

Bismuth subgallate is the product of the reaction between gallic acid, glacial acetic acid and bismuth nitrate and is represented by molecular formula of C₆H₂(OH)₃COOBi(OH)₂. It is known as an oral anti-diarrhea agent and a haemostatic agent. Being a benzene derivative, bismuth subgallate can also be used as a disinfectant.

Borneol was primarily known to be isolated from Dryobalanops araomatica or the like and its presented molecular formula is C₁₀H₁₇OH. Today, this compound can be synthesized in industry. Borneol has a special smell and thus can be used as a flavoring agent (see U.S. Pat. No. 4,983,394) or an inactive additive in medicaments (see e.g., U.S. Pat. No. 5,164,184, U.S. Pat. No. 5,190,757 and U.S. Pat. No. 5,593,691). Boreneol-derived esters have been commonly utilized in perfume manufacture. U.S. Pat. No. 4,931,475 has proposed the use of borneol as an active agent for treating gallstone. It is also known as an inhibitor of microsomal enzyme and to have an anti-fungus effect.

U.S. Pat. No. 6,232,341 B1 discloses a topical pharmaceutical composition for healing wound comprising borneol and bismuth subgallate. U.S. Pat. No. 6,514,960 B1 and USP 20030158255 A1 disclose the uses of bismuth subgallate for inhibiting the production of nitric oxide synthases (NOS) and preventing and/or reducing skin deterioration, respectively. U.S. Pat. No. 6,514,960 B1 and USP 20030158255 A1 also disclose that the administration of bismuth subgallate in combination with borneol may cause a synergistic effect. However, none of the prior art teaches or suggests that a combination of bismuth subgallate and borneol can be used to treat melanoma.

It is surprisingly found that the combination of bismuth subgallate and borneol is useful in the reduction of the number of melanoma cells and the inhibition of the proliferation and the migration of melanoma cells.

### SUMMARY OF THE INVENTION

One object of the invention is to provide a method for treating melanoma in a subject comprising administering to said subject an effective amount of bismuth subgallate and an effective amount of borneol.

Another object of the invention is to provide a method for reducing the number of melanoma cells and/or inhibiting the proliferation of melanoma cells in a subject comprising administering to said subject an effective amount of bismuth subgallate and an effective amount of borneol.

Still another object of the invention is to provide a method for inhibiting the migration of melanoma cells in a subject comprising administering to said subject an effective amount of bismuth subgallate and an effective amount of borneol.

A further object of the invention is to provide a combination for treating melanoma in a subject, which comprises an effective amount of bismuth subgallate and an effective amount of borneol, wherein said amounts of bismuth subgallate and borneol are sufficient to kill melanoma cells and/or inhibit the proliferation of melanoma cells.

Still another object of the invention is to provide a combination for inhibiting the migration of melanoma cells in a subject, which comprises an effective amount of bismuth subgallate and an effective amount of borneol, wherein said amounts of bismuth subgallate and borneol are sufficient to inhibit the migration of melanoma cells.

### BRIEF DESCRIPTION OF THE DRAWINGS

FIGS. 1A and 1B show the cytotoxic effect of various concentrations of bismuth subgallate (BSG) and borneol (BO) on A375 cells.

FIG. 2 shows the cytotoxic effect of various concentrations of bismuth subgallate (BSG) and borneol (BO) on HaCaT cells.

FIG. 3 shows the cell cycle effect of bismuth subgallate (BSG) and borneol (BO) on A375 cells.

FIG. 4 shows the migration inhibiting effect of bismuth subgallate (BSG) and borneol (BO) on A375 cells.

### DETAILED-DESCRIPTION OF THE INVENTION

The present invention relates to a method for reducing the number of melanoma cells and/or inhibiting the proliferation of melanoma cells by administering bismuth subgallate in combination with borneol, thereby melanoma in a subject can be treated.

The present invention also relates to a method for inhibiting the migration of melanoma cells by administering bismuth subgallate in combination with borneol.

The present invention further relates to a combination for treating melanoma, which comprises an effective amount of bismuth subgallate and an effective amount of borneol, wherein said amounts of bismuth subgallate and borneol are sufficient to reduce the number of melanoma cells and inhibit the proliferation of melanoma cells.

The present invention further relates to a combination for inhibiting the migration of melanoma cells, which comprises an effective amount of bismuth subgallate and an effective amount of borneol, wherein said amounts of bismuth subgallate and borneol are sufficient to inhibit the migration of melanoma cells.

The term “bismuth subgallate” as used herein refers to the compound represented by a chemical formula of C₆H₂(OH)₃COOBi(OH)₂.

The term “borneol” as used herein refers to the compound represented by a chemical formula of Cₗ₀H₁₇OH, which can be isolated from Dryobalanops araomatica or be obtained from chemical synthesis.

The term “treatment” or “treat” as used herein refers to the lessening or ameliorating of at least one abnormal or undesirable condition associated with melanoma.

The term “inhibition” or “inhibit” as used herein refers to decreasing, limiting, or blocking the action or function of a process.

The term “in combination with” as used herein refers to a method of treatment in which two or more treatments are applied collectively or according to specific sequence, such that they produce a desirable effect.

The term “effective amount” as used herein refers to an amount sufficient to reduce the number of melanoma cells, inhibit the proliferation of melanoma cell and/or inhibit the migration of melanoma cells so as to treat melanoma.

According to the invention, the effective amounts of the active ingredients used in the method and the combination of the present invention may be determined by medical practitioners or other skilled persons, and include the respective amounts disused in the prior art disclosing bismuth subgallate and borneol that are mentioned supra. In any event, a physician or a skilled person will be able to determine the actual amounts that will be most suitable for an individual. The amounts of the active ingredients may vary depending on the condition that is to be treated, as well as the age, weight, sex and response of the particular patient to be treated.

Preferably, the ratio of bismuth subgallate to borneol used in the methods and the combinations of the present invention ranges from about 5:1 to about 10:1. More preferably, the ratio of bismuth subgallate to borneol ranges from about 7:1 to about 10:1. Most preferably, the ratio of bismuth subgallate to borneol is about 7:1.

Apart from the above-mentioned active ingredients, the method and combination of the present invention may further comprise other conventional therapies or agents that are helpful in the treatment of melanoma, such as surgical operation, radiotherapy, chemotherapeutic agents, vaccines, devices and the agents disclosed in US 2002150589, EP 1222928, US 2003114398, US 2003181429, EP 1530636, WO 2005044191, US 2004029967, EP 1581204, MD 1965F, JP 2002241314 etc.

Bismuth subgallate and borneol used in the method or combination according to the invention can be formulated for topical, oral, parenteral or other mode of administration, and can be administered together, sequentially or separately.

For topical applications, the combination of the invention is preferably administered in the forms of paste, cream, ointment, gel paste, and the like. Oral applications are preferably administered in the forms of capsules, tablets and/or liquid formulations. Injectable sterile solutions, preferably oily or aqueous solutions, as well as suspensions, emulsions, or implants, including suppositories, are particularly suitable for parenteral application. Ampoules are convenient unit dosages.

Suitable pharmaceutical carriers used in the combination of the present invention include, but not limited to, water, salt solutions, alcohols, polyethylene glycols, gelatin, carbohydrates (such as lactose, amylose or starch), magnesium stearate, talc, silicic acid, viscous paraffin, fatty acid esters, hydroxymethylcellulose, polyvinyl pyrrolidone, and the like.

The following examples further illustrate the present invention, but are not intended to limit the scope of the present invention. The modifications and substitutions known to those skilled in the art are still within the scope and spirit of the present invention.

### EXAMPLE 1

Cytotoxicity Assay

A375 human melanoma cells (obtained from Food Industry Research and Development Institute, Hsinchu, Taiwan, R.O.C. under accession number CCRC 60039) and normal human keratinocyte (HaCaT) cells (obtained from Dr. Lin P., Institute of Medical & Molecular Toxicology, Chung Shan Medical University, Taichung, Taiwan) were used for the test.

A375 and HaCaT cells were incubated in 96-well plates (Corning Incorporated COSTAR® 3599) at a density of 3×10³ cells/well and 5×10³ cells/well, respectively. After 24 hours, the cells were treated with various concentrations of bismuth subgallate (BSG) and borneol (BO) for 72 hours. The cytotoxicity effect of BSG and BO was estimated by the relative viability (%) of the cells, which was determined by measuring the reduction of tetrazolium salt MTT using a Microplate reader (Thermo Lab systems Multishkan Spectrum) at a test wavelength of 550 nm and a reference wavelength of 690 nm. The relative viability is calculated by the following equation:
Relative Viability(%)=ODₜₑₛₜOD_{control}×100
The results are represented by the means plus standard errors (Mean±SE) of five repetitions.

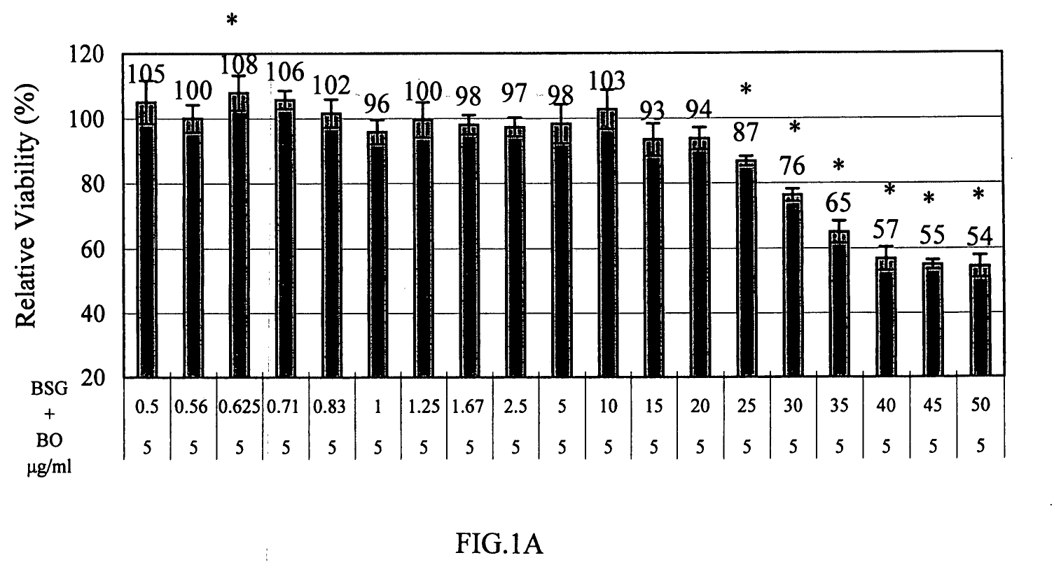

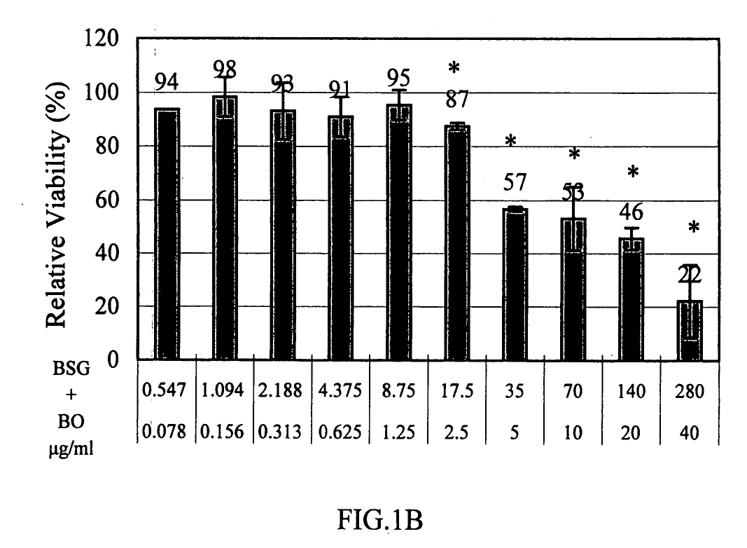

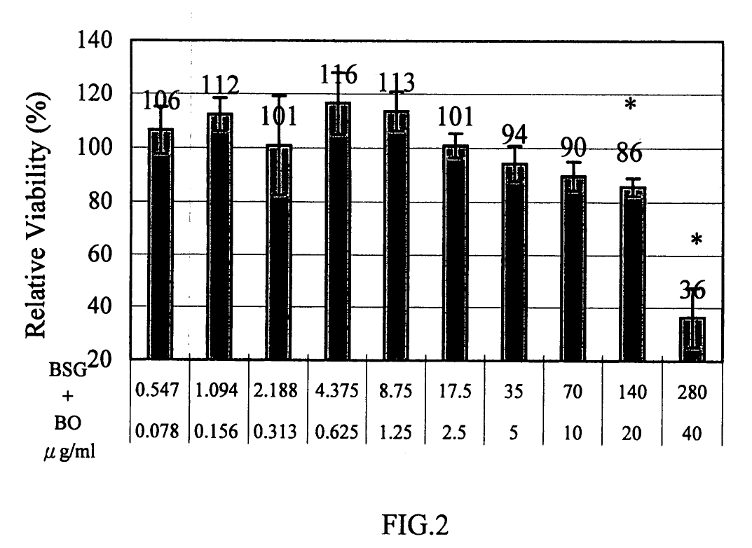

As shown in FIGS. 1A and 1B, the relative viability of A375 cells can be effectively reduced by various concentrations of BSG and BO, and the reducing effect can be observed at a concentration as low as 17.5 μg/ml for BSG and 2.5 μg/ml for BO. On the contrary, FIG. 2 shows that the cytotoxicity effect of BSG and BO on HaCaT cells can be observed only at high concentrations of BSG and BO, such as 140 and 280 μg/ml for BSG, and 20 and 40 μg/ml for BO. The above results suggest that the combination of BSG and BO has a cytotoxicity effect to the melanoma cells, but would not substantially affect the normal cells.

### EXAMPLE 2

Cell Cycle Assay

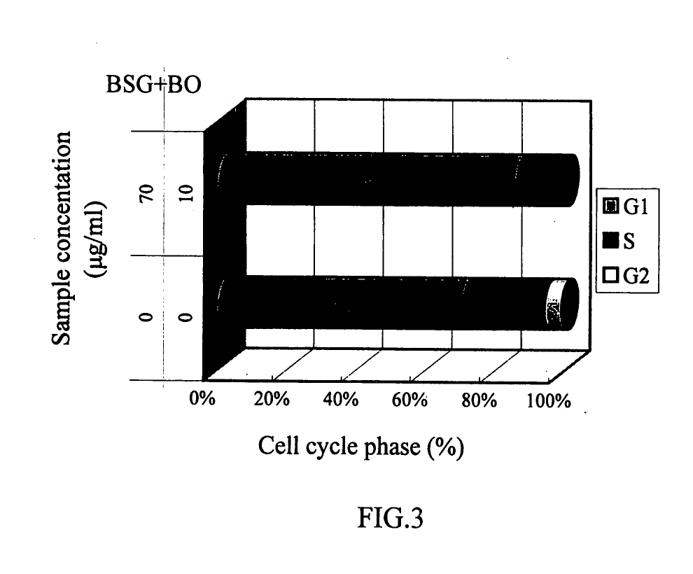

A375 cells were dispensed in a T25 flask at a density of 3×10⁵ cells. After 24 hours, the cells were treated with BSG (70 μg/ml) and BO (10 μg/ml) for 72 hours. For flow cytometry analysis, the cells were harvested and washed once by phosphate buffered saline (PBS). The cell pellets were fixed in 70% ethanol/PBS at 4° C. for at least 30 minutes, and then washed in PBS. The pellets were suspended in 1 ml of PI solution (40 μg/ml of Propoidum iodide (PI) and 100 μg/ml of Ribonuclease (Rnase) in PBS) and left at room temperature for 30 minutes. Cell samples were then incubated at 4° C. in the dark for at least 15 minutes and analyzed by a FACScan flow cytometer (Partec GmbH CyFolw®, Germany). The results are represented by the means values of three repetitions with standard errors (Mean±SE). As shown in FIG. 3, the combination of BSG and BO increase G1 phase arrest of A375 cells. This suggests that the combination of BSG and BO can inhibit the proliferation of melanoma cells.

### EXAMPLE 3

Migration Assay

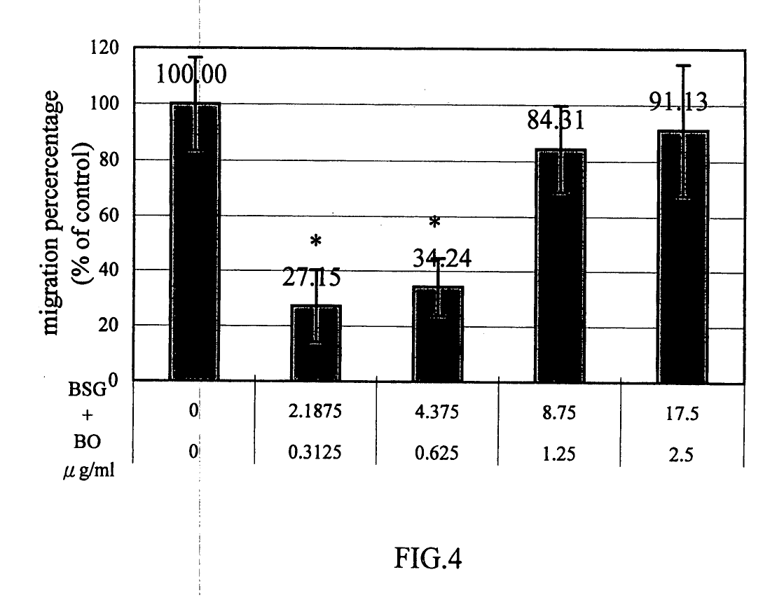

A375 cells were dispensed in 24-well Tran swell plates (Corning Incorporated COSTAR® Becton and Dickinson) at a density of 3×10³ cells/well and were treated with various concentrations of BSG and BO for 24 hours. After incubation, filters were fixed by 100% methanol, and then stained by hematoxylin (Sigma) for 30 minutes. The cells were observed and counted by light microscopy. The results are represented by the means values of nine repetitions with standard errors (Mean±SE). As shown in FIG. 4, the combination of BSG and BO can effectively inhibit the migration of melanoma cells.

What is claimed is:

1. A method for treating melanoma in a subject comprising administering to the subject an effective amount of bismuth subgallate in combination with an effective amount of borneol.

2. The method according to claim 1, wherein the ratio of bismuth subgallate to borneol ranges from about 5:1 to about 10:1.

3. The method according to claim 2, wherein the ratio of bismuth subgallate to borneol ranges from about 7:1 to about 10:1.

4. The method according to claim 3, wherein the ratio of bismuth subgallate to borneol is about 7:1.

5. A method for reducing the number of melanoma cells and/or inhibiting the proliferation of melanoma cells in a subject comprising administering to the subject an effective amount of bismuth subgallate and an effective amount of borneol.

6. The method according to claim 5, wherein the ratio of bismuth subgallate to borneol ranges from about 5:1 to about 10:1.

7. The method according to claim 6, wherein the ratio of bismuth subgallate to borneol ranges from about 7:1 to about 10:1.

8. The method according to claim 7, wherein the ratio of bismuth subgallate to borneol is about 7:1.

9. A method for inhibiting the migration of melanoma cells in a subject comprising administering to the subject an effective amount of bismuth subgallate in combination with an effective amount of borneol.

10. The method according to claim 9, wherein the ratio of bismuth subgallate to borneol ranges from about 5:1 to about 10:1.

11. The method according to claim 10, wherein the ratio of bismuth subgallate to borneol ranges from about 7:1 to about 10:1.

12. The method according to claim 11, wherein the ratio of bismuth subgallate to borneol is about 7:1.
